(19) United States
(12) Patent Application Publication
(10) Pub. No.: US 2003/0092971 A1
(43) Pub. Date: May 15, 2003

(54) Personal health monitoring system

(76) Inventor: Nathan Intrator, Providence, RI (US)

Correspondence Address: WEINGARTEN, SCHURGIN, GAGNEBIN & LEBOVICI LLP, TEN POST OFFICE SQUARE, BOSTON, MA, 02109, US

(21) Appl. No.: 09/993,324

(22) Filed: Nov. 12, 2001

(51) Int. Cl. A61B 5/00

(52) U.S. Cl. 600/300; 600/301; 128/903; 128/904

(57) ABSTRACT

The present invention includes a sensor, a basic processor and a complex processor. The basic processor resides with the sensor; it receives signals from the sensor and performs basic analysis. If conditions require further analysis, the basic processor communicates with the complex processor (the base unit) for further analysis. The base unit can alert the patient, a care giver, or can control the administration of medication. The basic processor can provide a more limited form of alert in case it is in a stand-alone mode.

The base unit enables bi directional communication between the sensor and the caregiver. The base unit can handle more than one sensor for a comprehensive medical and other vital signs monitoring.

### BACKGROUND

1. Field of the Invention

The present invention relates to equipment for monitoring physiological conditions of a user, and, more particularly, to portable equipment for continuous monitoring of conditions.

2. Discussion of Related Art

Systems for monitoring various physiological conditions are fairly common. Hospitals regularly use equipment to monitor respiration and heart performance. Such systems provide information useful for a caregiver to determine the necessary care. Thus, with an electrocardiograph (EKG) machine, a physician can review the operation of the patient's heart to determine irregularities. These systems can also process signals recorded by the monitor to activate an alarm when certain conditions occur. Thus, the system monitors the patient even while a caregiver is not present. However, such systems generally require the patient to remain relatively still and in one location. The need for extensive signal processing for adequate monitoring had required up to very recently, large devices, which used significant power and thus, large batteries. Such large and cumbersome devices are too heavy to be carried by a person for constant monitoring. There is a need however, for devices which can continuously monitor a user during ordinary life activities. Such devices must be very small, almost unnoticeable to provide the flexibility to be used by babies, children and elderly people in a clinical setup and during daily routines, including physical exercise.

U.S. Pat. No. 4,428,381 discloses a system, which attempts to allow more extensive movement through remote monitoring. That monitoring device converts sensed signals into digital forms, which are temporarily stored and transmitted through telephone lines to a processing computer. The sensor and transmitter do not monitor the signals; only the processing computer does the monitoring. Thus, the user has to decide when to connect the device to the telephone lines for monitoring.

Furthermore, most systems are utilized to diagnose acute conditions, rather than to provide advanced warnings and preventive advice. Some devices are used to determine the cause of certain symptoms already experienced by the patient. Most systems do not provide early warning at the onset of symptoms. For example, if the onset of an asthma attack can be detected, early intervention would significantly limit the severity and duration of the attack. Many conditions could be more easily addressed with early medical intervention. Another example is early warning of a heart attack that would allow the patient to be treated before symptoms become severe. Therefore, there exists a need for a system, which can provide early warnings at the onset of symptoms before they are recognized by the patient. Furthermore, a monitor which can provide an early warning of symptoms is useful for recognizing and treating chronic conditions in children, elderly people or other people risk e.g. in a bio-hostile environment. Often, these populations are not aware of or do not pay attention to early symptoms. They may be unable to recognize early symptoms or convey information to caregivers, parents or commanding officers, so that immediate action can be taken. Some portable health monitors have been developed to monitor various medical parameters. Some simply record specific data, while others provide an output to the patient, which is indicative of the physical parameters they sense. Some monitors simply provide an alarm when the parameters reach a pre-set level of particular concern. However, such systems lack the capability to store information for extended periods of time or to provide extensive processing or analysis of recorded signals.

U.S. Pat. No. 5,928,156 discloses a portable system for continuous monitoring respiratory sounds. Many simplifications have been made to the system in order to limit the weight and allow long term use. For example, the device is limited to monitoring a single, given characteristic frequency determined individually for the user for a specific conspicuous respiratory sound previously determined to have a significant feature. The device is operated only during short periods of time in a respiratory cycle to further limit power use. Although the device may activate an alarm based upon the simple comparisons being performed, it cannot provid extended analysis. Rather, data is generally just stored and can be analyzed later. As a result of the simple analysis done by the device, the alarm may be falsely activated or may miss the onset of certain symptoms. Thus, a need exists for a system with extensive processing capabilities.

### SUMMARY OF THE INVENTION

The deficiencies of the prior art are substantially overcome by the system of the present invention, which utilizes two separate components for sensing and recording physiological parameters. The invention includes a sensor and a basic processor. The basic processor which resides with the sensor determines possible irregular conditions through processing signals from the sensor. The basic processor may also store sensed data when certain conditions prevail. Under certain defined conditions, the information from the basic processor can be transferred, e.g., via short-range wireless transmission, to a complex processor. The complex processor further analyzes the information to determine whether certain conditions exist and whether additional actions should be taken. Either the basic processor and/or the complex processor can provide an alarm for the user to indicate when certain actions should be taken. Since large storage and processing capabilities are not required for the basic processor, it can be made small and portable. The connections to the complex processor need only be made when certain conditions are present, as determined by the basic processor.

One application of this system can be used to monitor respiratory sounds to determine the onset of wheezing which may indicate the onset of an asthma attack. The system can provide continuous monitoring of patients to provide an early warning system. In such a system, the basic processor may determine whether certain abnormalities of respiratory sounds are present. The complex processor can be used to further determine whether wheezing is commencing. Based upon the analysis, the user can perform certain treatments, or contact a physician. Similarly, the system can be used to monitor heart performance, or the alertness of a driver, or breathing cycle in babies and elderly people.

The complex processor can control several different sensors at once to provide a wider range of vital signs monitoring. This sensor which is best thought of as a handheld device with cellular communication capabilities, can also receive requests from caregiver or other monitoring officer to provide vital signs such as heart rate, blood pressure temperature etc, eliminating the need to send a nurse every few hours to perform those tests. The basic processor sensor can communicate bi-directional with the complex sensor to respond to complex sensors request and provide information about battery life etc. A mechanism exist to bind a basic processor/sensor to a complex sensor by connecting them for a short time so that they can pass code between them to set up the future communication only with a specific complex sensor. In this way, many patients can have many basic sensors on them communicating with the right complex sensor, and sensors can be shared between patients as needed upon binding them to the appropriate complex sensor. It should be emphasize that a system of this type can have additional sensor/processor blocks which monitor additional important conditions such as the level of oxygen in a patient's oxygen tank, the battery level in a patient's wheel chair the level of medication that is supplied to the patient, or even the level of gasoline and ammunition in a special operations unit. All these sensors can be wirelessly connected to one complex sensor which then responds to external requests, or communicates when certain conditions arise. Another important use of the present invention is in monitoring (in a closed loop) the supply of medication to a patient when certain conditions occur, e.g. asthma medication, reducing the amount of medication when conditions improve and alerting care givers when conditions do not improve. Information about the effectiveness of certain medication can then be deduced from the process.

### BRIEF DESCRIPTION OF THE DRAWINGS

FIG. 1 is a block diagram of a first embodiment of the invention of the present invention.

FIG. 2 is a block diagram of a second embodiment of the invention.

FIG. 3 is a block diagram of a third embodiment of the invention

### DETAILED DESCRIPTION

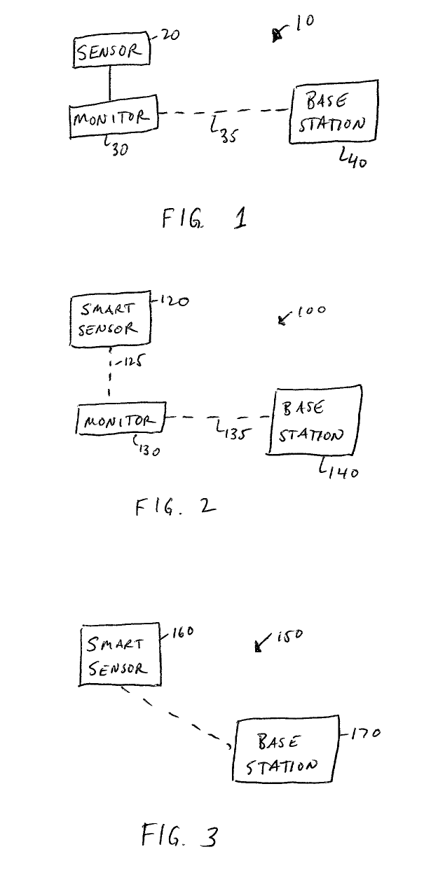

A portable system for data collection and analysis of medical signals such as heart or respiratory sounds, blood oxygen or pressure etc. has to be very small in order to avoid interference with patient's normal life style. The size requirement suggests that the computational engine, which analyzes the data, alerts the patient or the care giver or provides other monitoring or analysis, can not be situated at the location of the sensor as it is too big and bulky. However, connecting the computational engine to the sensor greatly limits the mobility of the patient. Therefore, according to the present invention, two different processing systems are used to record and analyze medical signals. As illustrated in FIG. 1, an embodiment of the present invention is a system 10 having a monitor 30 and a base station 40. The monitor 30 is connected by a wire 25 to a sensor 20 appropriately placed on the patient. For example, in monitoring asthma conditions, the sensor may be an acoustic microphone attached to the patient's chest to generate signals of breath sounds. The monitor 20, which is worn by the user, can be sized so as to be portable. It only requires sufficient power, processing capability and storage space to provide basic processing of the signals and transmission to the complex processor. By limiting the processing done by the monitor 30 and making the transmission data dependent, the power supply, which provides substantial weight for the device, can be limited. The base station 40 can remain in a fixed location.

The monitor 20 can be periodically connected to the base station 40 in order to transfer data. Various mechanisms 35 can be used to connect the monitor 30 to the base station 40. The type of connection may depend upon the nature and location of the base station 40. For example, the base station may be located in a doctor's office or hospital. The patient may be able to connect the monitor 30 to the base station 40 over telephone lines using a cellular or other type of modem. Alternatively, if the base station 40 were located within a room or a house of the patient, different wireless technologies, such as RF or infrared, could be used to transfer information from the monitor 30 to the base station 40.

During operation, the monitor 30 provides basic processing of signals from the sensor. Basic processing is used to determine whether certain conditions are present which suggest the need for further analysis. When conditions suggest, the monitor 30 provides information, either current or stored, to the base station 40. The monitor 30 may provide an alarm signal to the patient indicating the need to connect to the base station 40. Alternatively, when the monitor communicates wirelessly, the monitor 30 may automatically output a signal to the base station 40 whenever further analysis is needed. The base station 40 may include a single device or may include several devices at various locations, such as throughout a house. Alternatively, the base station 40 may have communication connections at various locations for connection with the monitor 30. With multiple base stations 40 or communication connections, cellular or other wireless technologies can be used to determine the appropriate connection method. Similarly, multiple monitors 30 could be used with a single base station 40 through different connections, either geographic or time based. In a controlled environment, such as in a home or hospital, a cellular system of base stations 40 may be implemented to communicate with monitors which move around in the system. Using systems similar to current cellular technology, the user could move from one base station coverage area to another.

FIG. 2 illustrates a second embodiment of the present invention which provides greater comfort to the user by eliminating the wire connecting the sensor to the monitor. The system 100 of the second embodiment includes a smart sensor 120 instead of a simple sensor 20 as used in the first embodiment. The smart sensor 120 uses wireless technology 125 to communicate with the monitor 130. The smart sensor includes its own battery and basic processing capability. As with the monitor 20 in the first embodiment, the smart sensor 120 can perform some basic processing of signals. Information can be communicated to the monitor 130 upon the occurrence of certain conditions. By communicating solely under limited conditions, power required by the smart sensor 120 can be reduced. Thus, the smart sensor can be made sufficiently small and light so that it can be comfortably worn by the user.

Preferably, the smart sensor 120 consists of a sensor for data collection, a low power digital signal processing (DSP) unit for initial processing of the data, a low range wireless communication of the (processed) data to the monitor 120 where the main data analysis storage and decisions are taking place. The low range wireless communication can be controlled by the DSP or the monitor using a blue-tooth or IEEE 802.11b type device. When possible, the transmission is done after data compression that is also performed by the DSP. A sensor has to be bound to a certain complex processor, so that there is a secured private communication channel only between the sensor and the appropriate complex processor, this is achieved by mounting the basic sensor shortly on the complex processor, so that the two can exchange the relevant key for secured communication between them. Thus, in an environment that is full of sensors and complex processors, there is no problem association between the sensor and the appropriate complex processor.

As with the first embodiment, the system 100 of the second embodiment includes a base station 140 which communicates 135 with the monitor 130. The same system discussed above can be used for the monitor 130 to communicate with the base station 140.

FIG. 3 illustrates a third embodiment 150 of the present invention which eliminates the monitor 130. The smart sensor 160 can communicate directly with the base station 170. This embodiment is most useful in controlled environments, where the smart sensor within maximum distances of a base station 40, so the wireless communications can be effective without significant power requirements. Additionally, power can be further conserved through control of operation of the smart sensor. For example, when conditions appear sufficiently normal to the basis sensor analysis algorithm, the smart monitor can reduce its frequency of data analysis. If the user leaves the area covered by the base station 170, the smart sensor will reduce communication effort to conserve energy. It should be stressed though, that as long as the basic sensor is activated, it will collect and analyse data and alert the user when needed. Thus, leaving the area covered by the complex sensor only partially reduces the performance of the smart sensor.

The complex processor can be equipped with a GPS for added information about the location of the user. This can be useful when a need arises to send emergency medical team to the patient or for various other strategic planning.

While the present invention has been described as monitoring physiologic parameters relevant to a medical condition, it can also be used in other situations where monitoring is useful within a controlled environment. For example, a smart sensor may be placed upon a driver with a base station located in the car. The smart sensor monitors some vital signs of alertness. When alertness appears degraded, information can be transferred to the base station in the car for further analysis. If the driver appears impaired upon further analysis, alarms can be activated so that the driver can correct the problem. As discussed above, the smart sensor may power up and down depending upon whether the driver is current in the car and communication with the base station are possible.

Having described at least one embodiment of the invention, those of skill in the art will readily understand that modifications and alterations can be made to the described systems without departing from the spirit and scope of the present invention. The invention is solely limited by the appended claims.

We claim:

1. A portable system for monitoring physiological conditions comprising:
a sensor attached to a user generating signals based upon conditions measured on the user;
a portable basic processing device which communicates with the sensor and performs a first analysis on the signals obtained from the sensor;
a base station receiving second signals from the basic processing device for performing asecond analysis of the second signals for determining a medical condition; and
wherein the basic processing device generates the second signals based upon the first analysis.

2. The portable system for monitoring physiological conditions according to claim 1, wherein the basic processing device communicates bi-directionally through a wire to the sensor to receive the signals.

3. The portable system for monitoring physiological conditions according to claim 1, wherein the sensor communicates bi-directionally with the basic processing device using a wireless technology.

4. The portable system for monitoring physiological conditions according to claim 1, wherein the basic processing device and the sensor are combined in a single unit worn by the system user.

5. The portable system for monitoring physiological conditions according to claim 1, wherein the second signals are communicated using a wireless technology.

6. The portable system for monitoring physiological conditions according to claim 1, wherein the second signals are communicated using a modem and telephone lines.

7. The portable system for monitoring physiological conditions according to claim 1, wherein the second signals are only sent when certain conditions are determined by the first analysis of signals.

8. The portable system for monitoring physiological conditions according to claim 1, wherein the base station generates an alarm based upon the second analysis of the second signals.

9. A portable system for monitoring physiological conditions comprising:
a sensor worn by a user having low power processing and communication capabilities; and
a base station receiving first signals from the sensor for performing an analysis of the first signals for determining a medical condition.

10. The portable system for monitoring physiological conditions according to claim 9, wherein the sensor performs an analysis of sensed data to generate the first signals.

11. The portable system for monitoring physiological conditions according to claim 10, wherein the sensor transmits the first signals only when certain conditions are determined by the analysis of sensed data.

12. The portable system for monitoring physiological conditions according to claim 9, further comprising a plurality of base stations, each of the base stations receiving signals from the sensor within a specified geographic area.

13. The portable system for monitoring physiological conditions according to claim 9, wherein the base station generates an alarm based upon the analysis.

14. The portable system for monitoring physiological conditions according to claim 9, wherein the sensor generates an alarm based upon processing of sensed data.

15. The portable system for monitoring for monitoring physiological conditions according to claim 9 further comprising:
at least one second sensor having low power processing and communication capabilities for monitoring a related parameter and providing second signals; and
wherein the base station receives the second signals, and performs an analysis of the first signals and second signals for determining a medical condition.

16. The portable system for monitoring physiological conditions according to claim 15, wherein the at least one second sensor includes a sensor for monitoring at least one of:
a medication level, an oxygen level, a battery level, and fuel level.

17. The portable system for monitoring physiological conditions according to claim 9, further comprising:
a plurality of sensors worn by the user at different locations, each of the sensors having low power processing and communication capabilities; and
wherein the base station receives a plurality of first signals from the plurality of sensors and performs an analysis of the plurality of first signals for determining a medical condition.
